(19) United States
(12) Patent Application Publication
(10) Pub. No.: US 2022/0305215 A1
(43) Pub. Date: Sep. 29, 2022

(54) RESUSCITATION DOSING KIT

(71) Applicant: Seattle Children's Hospital d/b/a Seattle Children's Research Institute, Seattle, WA (US)

(72) Inventors: Heather Anne Cruz, Seattle, WA (US); Jennifer Janett Cerdena, Seattle, WA (US)

(73) Assignee: Seattle Children's Hospital d/b/a Seattle Children's Research Institute, Seattle, WA (US)

(21) Appl. No.: 17/705,082

(22) Filed: Mar. 25, 2022

Related U.S. Application Data
(60) Provisional application No. 63/166,542, filed on Mar. 26, 2021.

(51) Int. Cl. A61M 5/34; A61M 16/04

(52) U.S. Cl. CPC A61M 5/345; A61M 16/0463; A61M 2205/6081; A61M 2205/6009; A61M 2005/3126

(57) ABSTRACT

A kit for resuscitating a subject, particularly a neonatal patient, is described. In an embodiment, the kit includes a syringe having a tip defining a male Luer taper fitting, and an adaptor defining a first end portion shaped to couple with the male Luer taper fitting and a second end portion shaped to cooperatively couple with an opening of an endotracheal tube.

### CROSS-REFERENCE(S) TO RELATED APPLICATION

This application claims the benefit of U.S. Provisional 63/166,542 filed Mar. 26, 2021, the entire disclosure of which is hereby incorporated by reference.

### BACKGROUND

Successful transition from intrauterine to extrauterine life is dependent upon several significant physiologic changes that occur at birth. In most cases, these changes are successfully completed at delivery without any outside intervention. However, nearly ten percent of the nearly 4 million infants born in the United States annually require some level of assistance to begin breathing at birth. Of these, approximately 1% require extensive resuscitation and about 0.3% develop significant morbidity and mortality due to hypoxic-ischemic brain injury. Mortality in infants with hypoxic-ischemic encephalopathy (HIE) ranges from 6-30%. Significant morbidity, such as cerebral palsy and long-term disabilities, occurs in 20-30% of survivors. The Neonatal Resuscitation Program (NRP) was developed in 1987 to identify infants at risk of significant morbidity and mortality and provide high-quality resuscitation. The NRP program has undergone several revisions for improvement over the years.

NRP has several prescribed steps in resuscitation focusing on airway, breathing, circulation, and medications. Several steps must be managed prior to the medication phase of the NRP pathway including adequate ventilation measures and chest compressions. Specifically, if the heart rate is less than 60 beats per minute despite adequate ventilation and chest compressions, epinephrine is administered via endotracheal tube (ETT) or more optimally the umbilical venous catheter (UVC).

The current standard for neonatal epinephrine delivery requires use of a system designed for adult dosing. In an adult code, the epinephrine carpuject syringe delivery system assembles quickly, and the entire prefilled syringe is used in a single dose per the American Heart Association's prescribed algorithm. However, this adult delivery system requires multiple steps, manipulations, and additional equipment in order to convert into an accurate neonatal dose.

Similarly, dosing and delivery complexities exist in delivering other rescue medications such as naloxone, lidocaine, vasopressin, atropine.

The current method of conversion from an adult dose into a neonatal dose causes significant delays in patient care due to cumbersome assembly and significant opportunity for errors in dosing. This current method requires a portion of the total adult dose to be withdrawn from the adult dosage carpuject syringe for delivery to the neonate. Additional opportunity for error is introduced by the fact that neonatal dosing is weight-based and the provider must first estimate a weight in the delivery room, complete a medication calculation, then withdraw the dose from the adult carpuject syringe.

As an example, the current preparation of epinephrine requires several pieces of equipment to be assembled to administer emergency dosing in a neonatal resuscitation. Currently, there is not an available prepared kit on the market that contains all required equipment. The required equipment is instead typically pieced together by the neonatal care team and available in a crash cart.

The American Academy of Pediatrics Neonatal Resuscitation Program suggests that epinephrine should be prepared in 10 seconds during a neonatal code. However, in practice, it can commonly take an otherwise skilled person several minutes to calculate a dose, assemble the equipment, prepare a syringe and have prescribed dose ready to administer to the patient. When considering stopping resuscitation efforts of a newborn around 10-15 minutes from birth, this is too long to wait for emergency administration of a lifesaving drug. It is unacceptable to have to wait for a dose of epinephrine during a code given the known immediate mortality and long-term morbidity outcomes.

Further complicating the matter, there is a difference in provider ability and comfort level in a neonatal resuscitation depending on the medical center in which a neonatal resuscitation occurs. For example, a center that handles thousands of deliveries a year with a high functioning NICU with an experienced code team with specialized providers will be much more prepared to rapidly prepare and calculate an accurate neonatal epinephrine dose. This is a different scenario in a smaller community hospital that may only have a few hundred deliveries a year of relatively healthy infants and rarely sees neonatal codes. There is even less resuscitation experience for deliveries that occur at a birthing center or in the community by emergency personnel. Additionally, the epinephrine dose may be prepared by a variety of providers (RN, ARNP, PA, MD, specialized neonatal pharmacist, hospital pharmacist, midwife, anesthesiologist, paramedic/EMT) all with varying levels of experience, skill, and competence. These factors contribute to the ability to accurately and quickly manipulate the current adult medication equipment to withdraw a potentially lifesaving medication.

Neonates are not born with a known weight or intravenous access in place. The delivery room weight is based on an estimate and the first available access for emergency medication is typically the airway. The estimated weight is based on provider experience, gestational age averages and estimated intrauterine fetal weight. These features present unique challenges to administering emergency medications accurately and efficiently to a neonate.

In a neonatal delivery room code, the first round(s) of epinephrine are given via the ETT. If the response is inadequate, then a UVC is emergently placed for IV dosing. The dosing between these two delivery modes have drastically different dosing up to 10-fold.

In current practice, the same epinephrine vial (1:10,000) is used to draw up the dose for both ETT and intravenous/intraosseous (IV/IO) dosing. Accurate dosing depends on the provider who is preparing the dose to know the different ETT and IV/IO dosing and/or use a dosing chart. The available dosing charts have multiple categories and are challenging to find the correct weight and then correlate to either ETT or IV/IO dosing.

Accordingly, there is presently a need for a kit suitable to quickly and accurately provide doses of resuscitating medications, particularly for neonates.

### SUMMARY

Toward this end, the present disclosure provides kits for resuscitating a subject, particularly a neonatal patient, to address these challenges detailed in the background, and related issues. In particular, the present disclosure provides kits that address multiple problems in existing neonatal resuscitation procedures including but not limited to: reducing or eliminating the need for equipment assembly (such as stop cock connectors) to allow for emergency medication delivery; eliminating potentially erroneous drug dosing calculations; facilitating direct endotracheal tube (ETT) administration, which is often first route of administration and reduces likelihood of mistaken intravenous/intraosseous (IV/IO) dosing with an ETT level dose of a drug; and safely allowing for more accurate and timely dosing of resuscitation drugs, such as epinephrine, to a neonate (babies <5 kg). As discussed further herein, even experienced personnel are generally unable to meet NRP timeliness guidelines with existing approaches and the complexities of dose calculation and manipulation can lead to errors.

Accordingly, in an aspect, the present disclosure provides a kit comprising a syringe having a tip defining a male Luer taper fitting, and an adaptor defining a first end portion shaped to couple with the male Luer taper fitting and a second end portion shaped to cooperatively couple with an opening of an endotracheal tube. In an embodiment, the first end portion of the adaptor comprises a female Luer taper fitting configured to cooperatively couple with the male Luer taper fitting, where the second end portion comprises a barrel portion shaped to couple to an outer surface of an endotracheal tube injection line, and a tube portion positioned coaxially within the barrel portion and shaped to couple with an inner surface of the endotracheal tube injection line. In an embodiment, the adaptor is configured to transfer liquid between the syringe coupled to the first end portion and an endotracheal tube coupled to the second end portion.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of the claimed subject matter will become more readily appreciated as the same become better understood by reference to the following detailed description, when taken in conjunction with the accompanying drawings, wherein:

FIG. 1 schematically illustrates the use of conventional equipment and medication for resuscitating a subject;

FIG. 2A is a perspective view of an adaptor coupled to a syringe, in accordance with an embodiment of the present disclosure;

FIG. 2B is another perspective view of the syringe and the adaptor of FIG. 2A shown separated, in accordance with an embodiment of the present disclosure;

FIG. 2C is a cross-sectional view of the adaptor of FIG. 2A, in accordance with an embodiment of the present disclosure;

FIG. 3A is a perspective view of an adaptor, in accordance with an embodiment of the present disclosure;

FIG. 3B is another perspective view of a syringe with the adaptor of FIG. 3A shown separated, in accordance with an embodiment of the present disclosure;

FIG. 3C is a cross-sectional view of the adaptor of FIG. 3A, in accordance with an embodiment of the present disclosure;

FIG. 4A is a perspective view of an adaptor, in accordance with an embodiment of the present disclosure;

FIG. 4B is another perspective view of a syringe with the adaptor of FIG. 4A shown separated, in accordance with an embodiment of the present disclosure;

FIG. 4C is a cross-sectional view of the adaptor of FIG. 4A, in accordance with an embodiment of the present disclosure;

FIG. 5A is a perspective view of an adaptor, in accordance with an embodiment of the present disclosure;

FIG. 5B is a perspective view of a syringe with the adaptor of FIG. 5A shown separated, in accordance with an embodiment of the present disclosure;

FIG. 5C is a cross-sectional view of the adaptor of FIG. 5C, in accordance with an embodiment of the present disclosure;

FIG. 6 illustrates an example of written indicia for administering a dose of a pharmaceutical composition to a subject in need thereof, in accordance with an embodiment of the present disclosure;

FIGS. 7A-7B illustrate examples of written indicia for administering a dose of a pharmaceutical composition for endotracheal use, in accordance with an embodiment of the present disclosure;

FIGS. 8A-8B illustrate examples of written indicia for administering a dose of a pharmaceutical composition for intravenous use, in accordance with an embodiment of the present disclosure; and

FIG. 9 schematically illustrates a kit and an example of its use, in accordance with an embodiment of the present disclosure.

### DETAILED DESCRIPTION

While illustrative embodiments have been illustrated and described, it will be appreciated that various changes can be made therein without departing from the spirit and scope of the invention.

Current protocols for delivering epinephrine and other resuscitation drugs to a neonate during an emergency require the use of an adult dose delivery system that is adapted and converted into a neonatal dose. These protocols require multiple equipment-assembly and dose-calculation steps that often result in delay in drug delivery and also lead to dosing errors. To address these challenges, the present disclosure provides kits for resuscitating a subject, such as a neonatal subject. The kits disclosed herein are a major advance on existing kits and protocols, with a focus on improving dosing safety and eliminating delays in neonatal resuscitation.

In an embodiment, the kits of the present disclosure include a syringe, such as a syringe for carrying and delivering a dosage of a liquid medication, and an adaptor coupled or couplable to an end of the syringe.

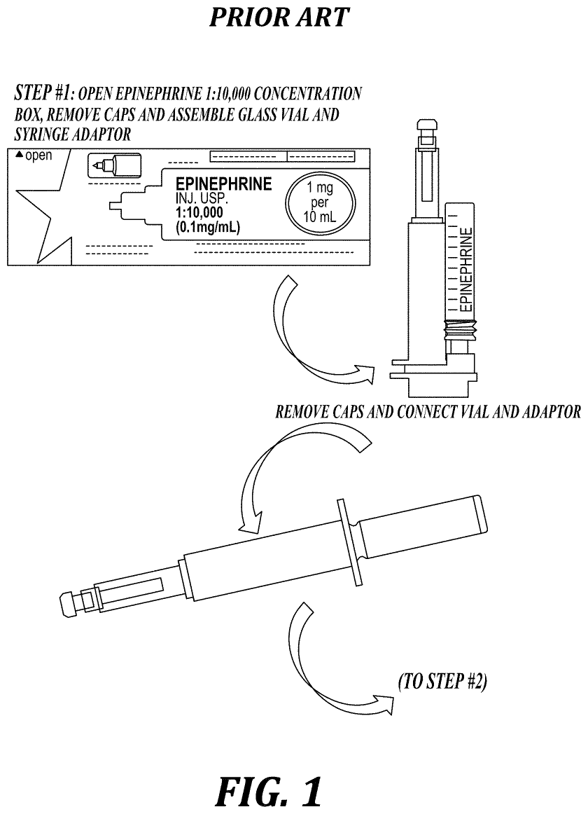

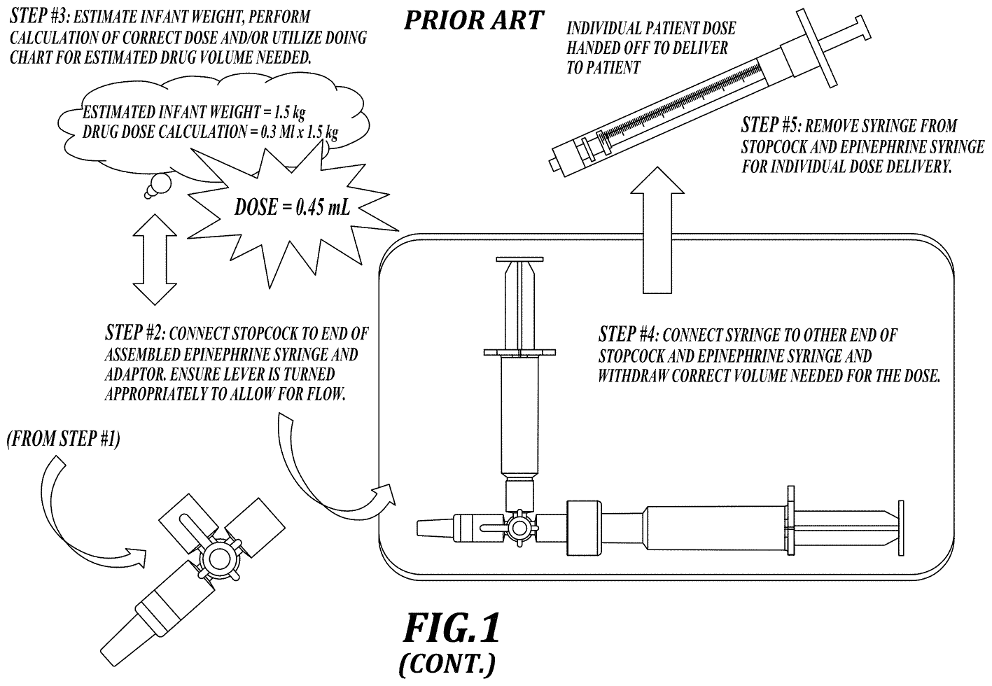

FIG. 1 schematically illustrates the use of conventional equipment and medication for resuscitating a subject. The following pieces of equipment and steps are conventionally used in resuscitating a neonatal patient:
1. Box of epinephrine 1 mg/10 mL (alternately labeled 0.1 mg/mL or 1:10,000), which typically contains a manufacturer supplied two-part vial/syringe system [“carpuject”] for adult dosing, comprised of the medication glass vial and needle/Luer adaptor
2. Three-way stopcock or other transfer device
3. Luer-lock dosing delivery syringes
4. Dosing chart, or calculator if dosing chart unavailable; the dosing chart is not standardized, typically provided by the healthcare team, and varies from location to location

As shown in FIG. 1, the procedure to draw up an epinephrine dose is multi step. In step #1, a practitioner must open the epinephrine box containing 1:10,000 concentration of epinephrine. The practitioner also must remove two caps from the epinephrine vial and assembly a glass vial and a delivery needle adaptor. The manufacturer delivery needle adaptor must also be screwed into the epinephrine vial. In step #2, the three-way stopcock or other transfer device needs to be attached to the syringe used for delivery. The lever on the stopcock must also be turned appropriately to allow for flow through the stopcock.

In step #3, the practitioner must manually calculate a dose or choose a dose from multiple categories found on a Neonatal Epinephrine Dosing Chart. For example, as shown in FIG. 1, the practitioner may have to perform a calculation using an estimated weight of the infant (or neonate), and a drug dosage calculation. After performing the calculation, the practitioner must then connect the syringe to the other end of the stopcock and epinephrine syringe and withdraw the correct volume needed for this dose, in step #4. Finally, in step #5, the syringe must be removed from the stopcock and epinephrine syringe for individual dose delivery. This dose is then handed off to deliver the drug to a patient.

As mentioned herein, it is recommended by The American Academy of Pediatrics Neonatal Resuscitation Program that epinephrine should be prepared in 10 seconds during a neonatal code. The conventional process can be extremely difficult for a practitioner to perform in that time limit, especially because the pieces required do not exist in a single kit, and the practitioner needs to perform a calculation that is not standardized between hospitals, or sometimes even between individual healthcare teams.

In an embodiment, the kits of the present disclosure include a syringe, such as a syringe for carrying and delivering a dosage of liquid medication, and an adaptor, coupled or couplable, to an end of the syringe.

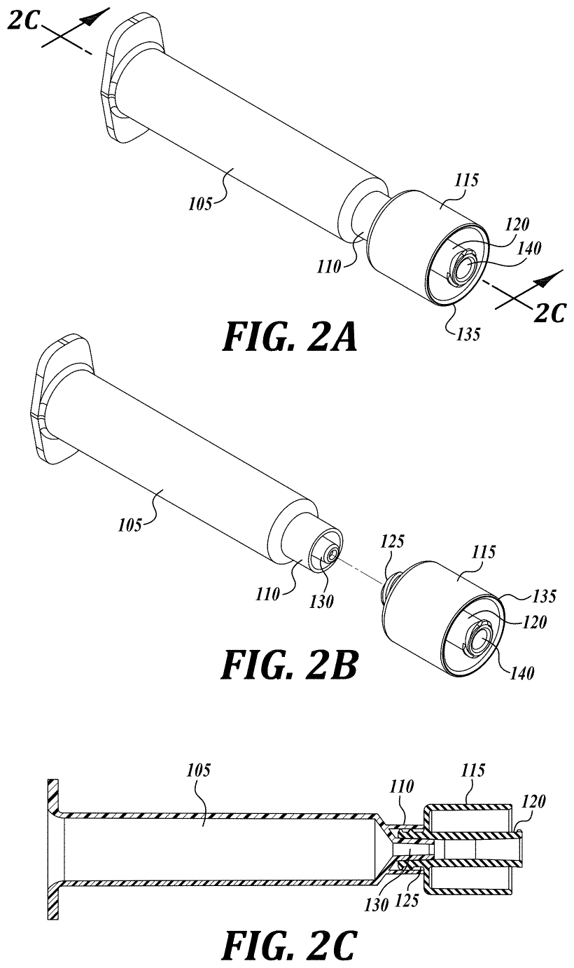

FIG. 2A is a perspective view of a kit 100 including an adaptor 115, in accordance with an embodiment of the present disclosure. In some embodiments, the syringe 105 has a tip 110 defining a male Luer taper fitting 130 (as illustrated in FIG. 2B), and an adaptor 115 defining a first end portion 125 shaped to couple with the male Luer taper fitting. While Luer taper fittings are described herein, it will be understood that other cooperative coupling structure are within the scope of the present disclosure. FIG. 2A shows an adaptor 115 and a syringe 105 cooperatively coupled thereto. In some embodiments, and as illustrated in FIG. 2A, the adaptor 115 is cylindrically shaped. In some embodiments, the adaptor 115 has a second end portion 120, comprising a barrel portion 135 and a tube portion 140.

FIG. 2B is a perspective view of the syringe 105 with the adaptor 115 separated, in accordance with an embodiment of the present disclosure. In the illustrated embodiment, the adaptor 115 is separable from the syringe 105. In some embodiments, the adaptor 115 includes a first end portion 125 shaped to couple with the tip 110 of the syringe 105. In some embodiments, the tip 110 of the syringe 105 is a male Luer taper fitting of the syringe 130. In an embodiment, the kit 100 further includes a syringe plunger (not shown) shaped to be slidably received by the syringe 105 and configured to urge fluid disposed in the syringe 105 through the adaptor 115 when coupled thereto.

As shown, the adaptor 115 further defines a second end portion 120 shaped to cooperatively couple with an opening of an endotracheal tube (not shown). The second end portion 120 of the adaptor 115 may also be shaped to incorporate an integrated male Luer taper fitting within the endotracheal tube coupling. This integrated male Luer taper fitting provides the adaptor 115 with dual functionality so that it can alternately be used to cooperatively couple with another Luer-compatible device (such as a syringe) rather than an endotracheal tube. In some embodiments the second end portion 120 includes a barrel portion 135 shaped to couple to an outer surface of an endotracheal tube injection line, and a tube portion 120 positioned coaxially within the barrel portion 135 and shaped to couple with an inner surface of the endotracheal tube injection line. In some embodiments, the second end portion 120 is shaped to couple to an inner surface of the endotracheal tube injection line or cuff.

While a separate adaptor 115 couplable to both a syringe 105 and an ETT injection line is described, it will be understood that a syringe 105 permanently affixed and/or integrated with the functionality of the adaptor 115 (to form the combined unit illustrated in FIG. 2A) is within the scope of the present disclosure. In some embodiments, the adaptor 115 is integrated directly into the syringe design, so that the adaptor 115 and the syringe 105 are a single piece.

FIG. 2C is a cross-sectional view of the adaptor 115, taken along the line 2C in FIG. 2A, in accordance with an embodiment of the present disclosure. The cooperative coupling between the adaptor 115 and the endotracheal tube injection line is suitable to provide liquid communication between the syringe 105 and the endotracheal tube (not shown in FIGS. 2A-2C). Accordingly, in an embodiment, the adaptor 115 is configured to transfer a fluid, such as liquid, between the syringe 105 coupled to the first end portion 125 and an endotracheal tube coupled to the second end portion 120 of the adaptor 115. In this regard, when the syringe 105 is coupled to an endotracheal tube injection line through the adaptor 115, a user can administer a dose of a pharmaceutical composition to a subject, such as a pharmaceutical composition disposed within a barrel of the syringe 105, through the endotracheal tube.

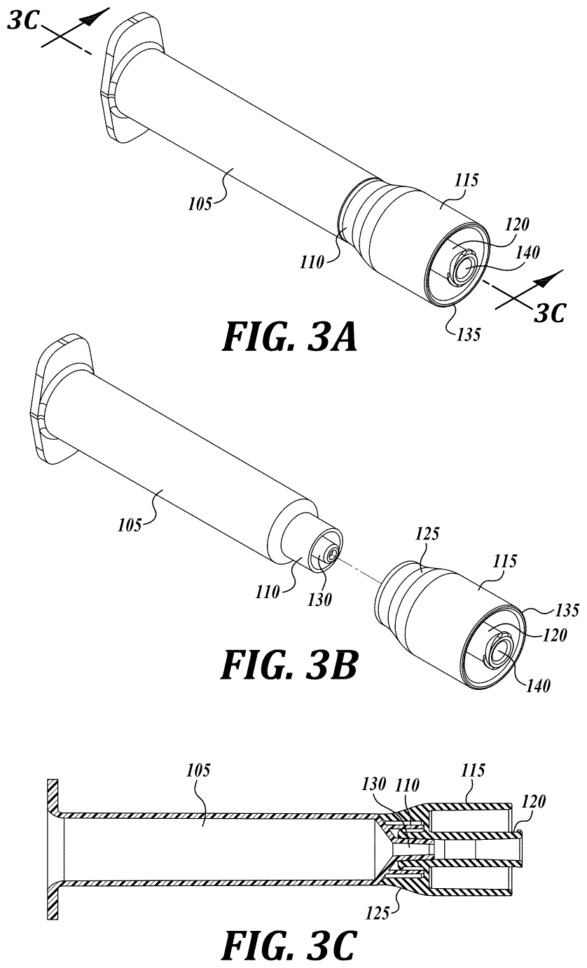

FIG. 3A is a perspective view of an adaptor 115, in accordance with an embodiment of the present disclosure. In some embodiments, the adaptor 115 is cylindrical and covers the male Luer taper fitting 130 of the syringe 105. In some embodiments, the adaptor 115 has a diameter equal to that of the syringe 105, that enlarges to form a second end portion 120 comprising a barrel portion 135 and a tube portion 140 for coupling to an endotracheal tube.

FIG. 3B is a perspective view of the syringe 105 with the adaptor 115 separated, in accordance with an embodiment of the present disclosure. As described previously, in some embodiments, the syringe 105 and the adaptor 115 are integrated to form a single piece. In some embodiments, the second end 120 includes a barrel portion 135, and a tube portion 140 for attaching to an endotracheal tube or a syringe. The syringe 105 also includes a tip 110 with a male Luer taper fitting 130.

FIG. 3C is a cross-sectional view of an adaptor 115, taken along the line 3C in FIG. 3A, in accordance with an embodiment of the present disclosure. Covering the male Luer taper fitting 130 strengthens the connection between the adaptor 115 and the syringe 105. It may also prevent a practitioner from accidentally pinching themselves or an object, for example, clothing or medical equipment, in the connection when working quickly. This configuration also provides the affordance that the adaptor is an integral part of the syringe, making it appear as a one-piece design. This may also prevent a practitioner from accidentally removing the adaptor.

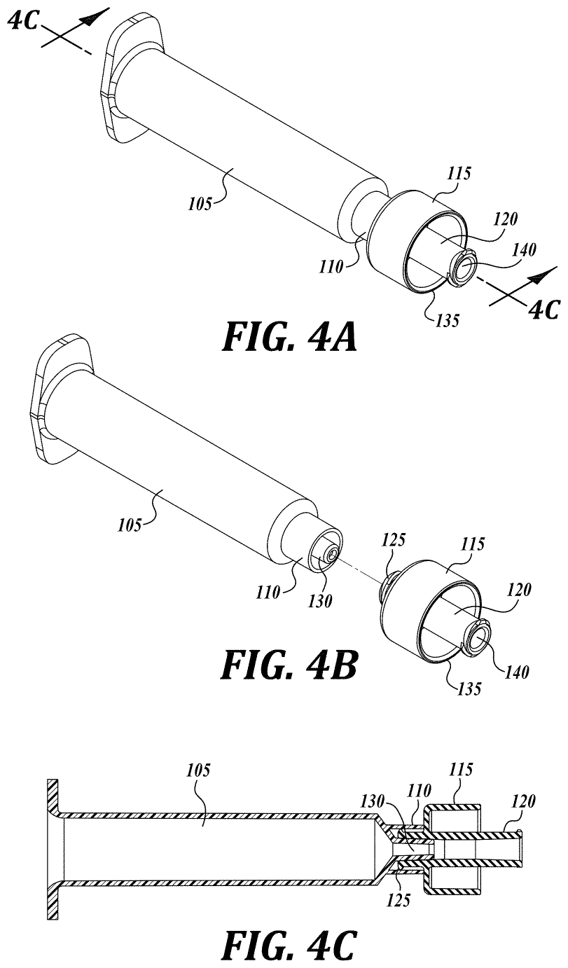

FIG. 4A is a perspective view of an adaptor 115, in accordance with an embodiment of the present disclosure. In some embodiments, the barrel portion 135 on the second end portion 120 does not extend the full length of the tube portion 140, so that the tube portion 140 extends past the barrel portion 135.

FIG. 4B is a perspective view of the syringe 105 with the adaptor 115 separated, in accordance with an embodiment of the present disclosure. In some embodiments, the syringe 105 includes an end portion 110 including a male Luer taper fitting 130. FIG. 4C is a cross-sectional view of an adaptor 115, taken along the line 4C in FIG. 4A, in accordance with an embodiment of the present disclosure. In some embodiments, the adaptor 115 is a thin cylinder that exposes more of the tube portion 140 of the second end portion 120. In some embodiments, the ratio between the adaptor 115 and the second end portion 120 is between 1:1.5 and 1:3. In some embodiments, the ratio between the adaptor 115 and the end portion 120 is 1:2. In some embodiments, the second end portion 120 is a male Luer taper fitting configured to couple with an endotracheal tube or a syringe 105. By leaving the tube portion 140 more exposed with the shorter barrel portion 135, the tube portion 140 can be more easily couplable to a syringe. Further, it may also allow the tube portion 140 to extend farther down the opening of an endotracheal tube or endotracheal tube connected to get closer to the endotracheal tube. The extended tube portion can provide a tighter fit with the inlet of an endotracheal tube and thereby improve the injection of the desired pharmaceutical composition into the lungs of a patient.

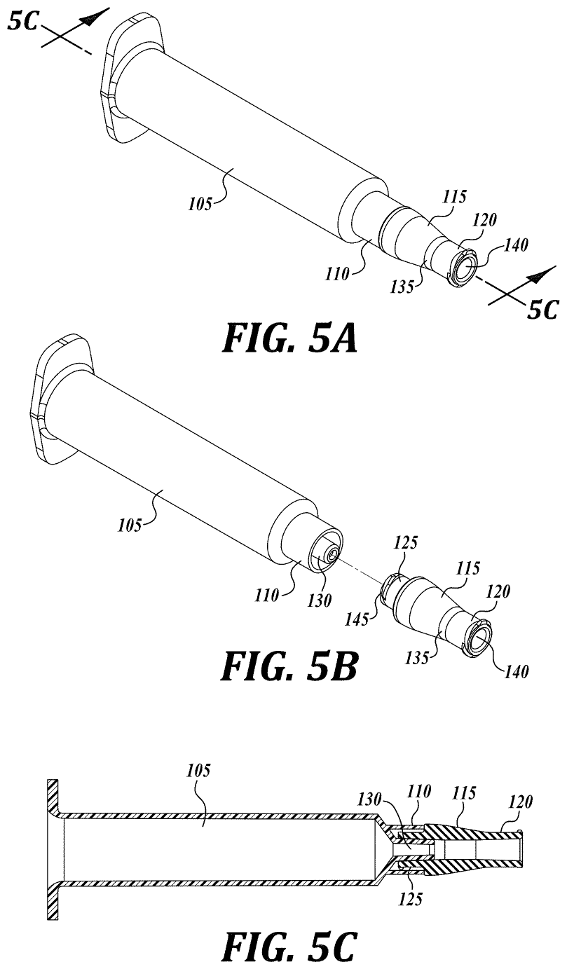

FIG. 5A is a perspective view of an adaptor 115, in accordance with an embodiment of the present disclosure. In some embodiments, the adaptor 115 may be about the same circumference of the male Luer taper fitting 130 of the syringe 105. In some embodiments, the adaptor 115 tapers down from a diameter of the tip of the syringe 105, to the diameter of the tube portion 140 of the end portion 120. In some embodiments, the barrel portion 135 of the adaptor may not surround the tube portion 140. In some embodiments, the barrel portion 135 extends the length of the second end portion 120. In some embodiments, the adaptor 115 may have no barrel portion.

FIG. 5B is a perspective view of a syringe 105 with the adaptor 115 separated, in accordance with an embodiment of the present disclosure. In some embodiments, the syringe 105 includes a tip 110 with a male Luer taper fitting 130. FIG. 5C is a cross-sectional view of an adaptor 115, taken along the line 5C in FIG. 5A, in accordance with an embodiment of the present disclosure. When the adaptor 115 does not have a barrel portion 135 surrounding the tube portion 140 of the second end portion 120, it may be easier to couple the adaptor 115 to a syringe 105, but may still be coupled to an endotracheal tube.

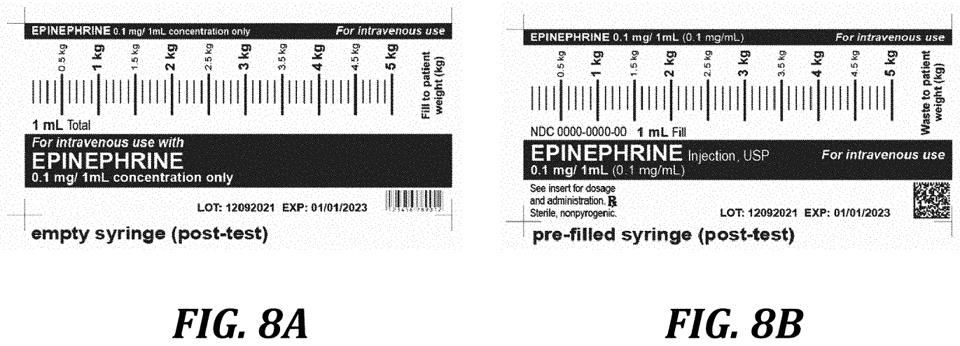

In some embodiments, the kit includes a first syringe and a second syringe and a first and second adaptor, as illustrated in FIG. 8. The two adaptors may have the same shape, such as a first and second adaptor shaped like the adaptor 115 in FIGS. 3A-3C. In some embodiments, the first syringe may have an adaptor shaped in first way, such as the shape of the adaptor 115 in FIGS. 4A-4C, and the second syringe may have an adaptor shaped in a second way, such as an adaptor in the shape of the adaptor 115 in FIGS. 5A-5C. In some embodiments, the second syringe may not include an adaptor. In some embodiments, the first syringe may have an adaptor with a second end shaped to couple to an endotracheal tube, while the second syringe may have an adaptor with a second end that is a male Luer taper fitting, configured to couple to a syringe.

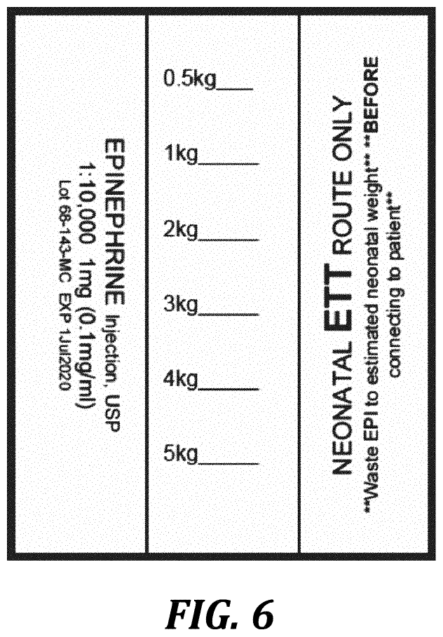

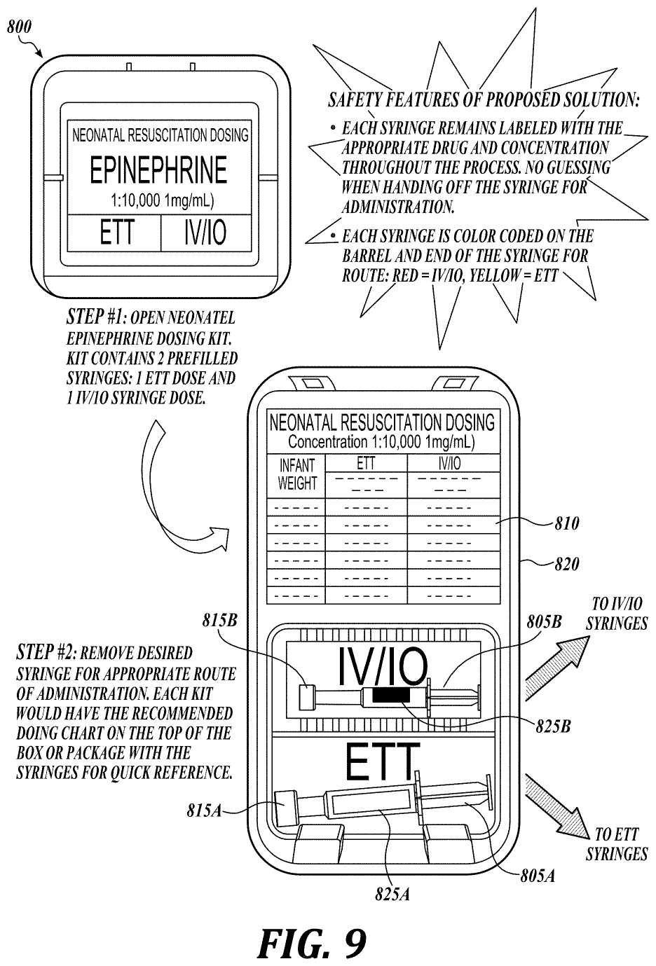

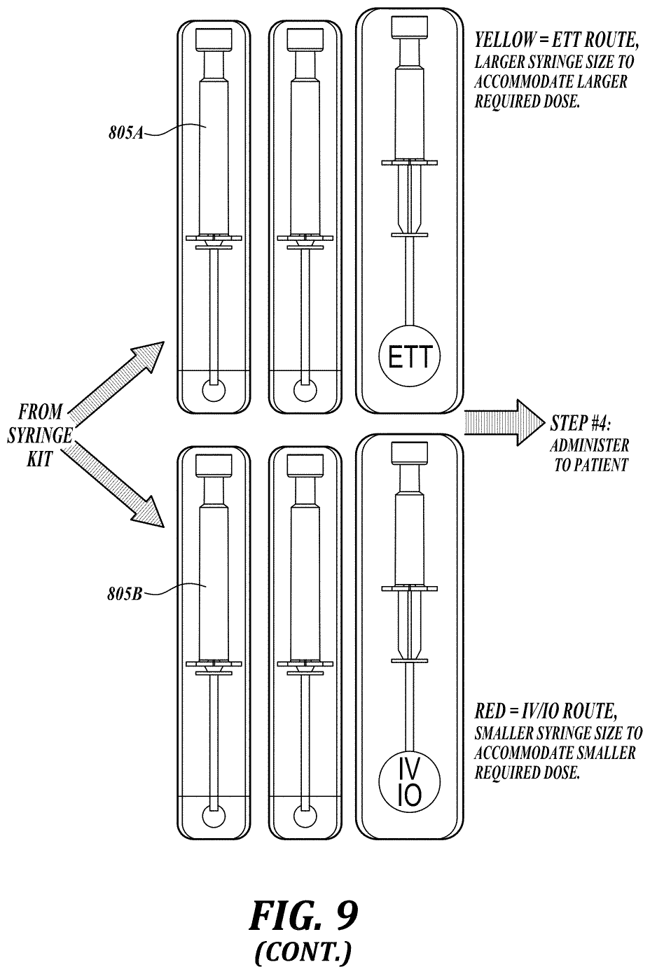

FIG. 6 illustrates an example of written indicia for administering a dose of a pharmaceutical composition to a subject in need thereof, in accordance with an embodiment of the present disclosure. As shown, the written indicia include subject weight range information from 0.5 kg to 5 kg. In an embodiment, the subject weight range information includes a plurality of ranges of subject weights, where the dosages corresponding to the subject weight range information and include recommended dosages corresponding to weight ranges in the plurality of ranges of subject weights. In some embodiments, the dosage is recommended visually by a line next to the subject weight, as illustrated in FIG. 6. In some embodiments, the written indicia are located on one or more instruments in the kit (as shown in FIG. 9). In some embodiments, the written indicia are located on the barrel of the syringe, such as syringe 105. In operation, a practitioner can administer a correct dose by pressing the plunger of the syringe until it reaches the marking indicating the present subject's weight. For example, if a patient weighed 3 kg, the practitioner would press the plunger until it reached the line reading 3 kg on the written indicia on the barrel of the syringe.

Some conventional instructions with dosages provide individual weights or narrow weight ranges corresponding to a pharmaceutical dose. In an emergent situation, it may be challenging to determine a dosage corresponding to the individual weight or narrow weight range in a timely fashion. This may be doubly challenging with a neonatal subject or other subject where a precise subject weight is not known. Accordingly, by providing subject weight range information includes recommended dosages corresponding to weight ranges in the plurality of ranges of subject weights, a health care provider can determine an appropriate and/or recommended dosage based upon an estimated weight.

TABLE 1
Simplified Dosing Chart.
NEONATAL RESUSCITATION EPINEPHRINE DOSING
Based on Concentration of 0.1 mg/mL (1:10,000)

| | ETT | Actual | IV/IO | Actual |
|---|---|---|---|---|
| NEONATAL | VOLUME | Weight-Based | VOLUME | Weight-Based |
| WEIGHT | [symbol] | [symbol] | [symbol] | [symbol] |
| 0.5 kg-0.9 kg | 0.5 mL | 0.6-1 mL/kg | 0.15 mL | 0.16-0.3 mL/kg |
| | (ETT route) | | (IV/IO route) | |
| 1-1.9 kg | 1 mL | 0.5-1 mL/kg | 0.3 mL | 0.16-0.3 mL/kg |
| | (ETT route) | | (IV/IO route) | |
| 2-2.9 kg | 2 mL | 0.7-1 mL/kg | 0.6 mL | 0.2-0.3 mL/kg |
| | (ETT route) | | (IV/IO route) | |
| 3-3.9 kg | 3 mL | 0.75-1 mL/kg | 0.9 mL | 0.23-0.3 mL/kg |
| | (ETT route) | | (IV/IO route) | |
| 4-4.9 kg | 4 mL | 0.8-1 mL/kg | 1.2 mL | 0.25-0.3 mL/kg |
| | (ETT route) | | (IV/IO route) | |
| 5-5.9 kg | 5 mL | 0.85-1 mL/kg | 1.5 mL | 0.25-0.3 mL/kg |
| | (ETT route) | | (IV/IO route) | |

[symbol]

indicates data missing or illegible when filed

In an embodiment, the kit includes written instructions further including dosages of the pharmaceutical composition corresponding to the subject weight range information and a method of administering the pharmaceutical composition. In some embodiments, the written instructions are fixed weight-based dosing conversion. In some embodiments, the fixed weight-based dosing conversion is 1 mg/kg of epinephrine delivered via ETT, and 0.2 ml/kg of epinephrine for IV/IO. The written instructions may change overtime to account for NRP recommendations. In some embodiments, the ratios shown in the written instructions may be any appropriate dosing ratio. As shown in Table 1, the written instructions include methods of administration including administering the pharmaceutical composition through an endotracheal tube and through an intraosseous line and/or through an intravenous line. In some embodiments, the written instructions are on the barrel of the syringe (such as syringe 105) in place of or in addition to the written indicia. In some embodiments, the written indicia are located on the barrel of the syringe, and the written instructions are located elsewhere in the kit, as further described in FIG. 9.

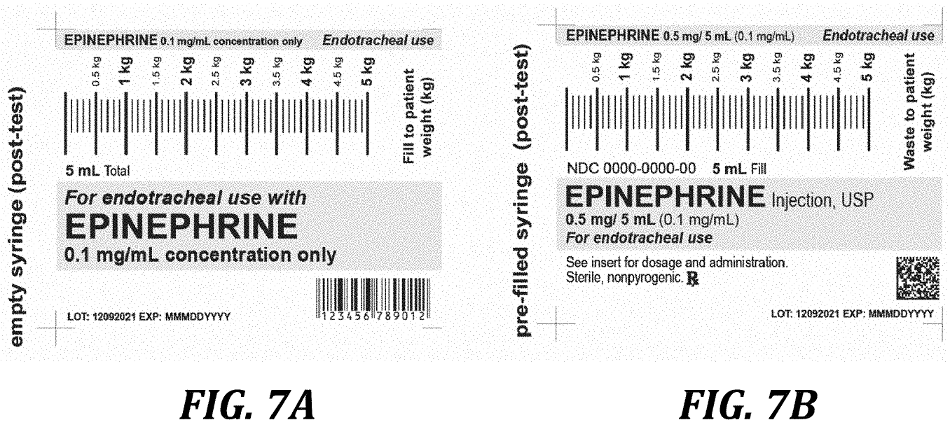

FIGS. 7A-7B illustrate an example of written indicia for administering a dose of a pharmaceutical composition for endotracheal use, in accordance with an embodiment of the present disclosure. In some embodiments, the kit described herein includes a first syringe and a second syringe, as shown in FIG. 9. In some embodiments, the first syringe is configured for endotracheal use.

FIG. 7A is an example of written indicia for the barrel of an empty syringe. In some embodiments, the kit includes an empty syringe configured to be filled with a pharmaceutical composition, such as epinephrine. Illustrated is an example dosing chart, and an indicator requiring a practitioner to use a specific concentration of epinephrine with the syringe. Although the example instructions indicate that 0.1 mg/ml concentration of epinephrine is required for use with the first syringe, in some embodiments, any concentration of epinephrine suitable to administer a dose to a patient may be indicated. The written indicia further include a range of patient weights from 0.5 kg to 5 kg in increments of half a kilogram. As explained in FIG. 6, these weights correspond to a dosage of the pharmaceutical composition, so that when a practitioner presses a plunger on the syringe up to the line next to one of the weights, the dosage for a patient of that weight is administered to the patient.

FIG. 7B is an example of instructions for administering a pharmaceutical composition for endotracheal use for a syringe that is prefilled. In some embodiments, the syringe is prefilled with a pharmaceutical composition. In some embodiments, the syringe is filled with 0.1 mg/ml concentration epinephrine. The prefilled syringe may have enough inside to administer a single dose to a neonate up to 5 kg, but in some embodiments, the prefilled syringe is prefilled with enough pharmaceutical composition for delivering the correct dose to a patient of any weight.

In an embodiment, the first syringe has a first color, and the second syringe has a second color, distinct from the first color. The first color and second color are suitable for further differentiating the differences and different intended uses of the first and second syringes. As shown, the first syringe intended for endotracheal tube delivery is labelled with gray markings in FIGS. 7A-7B. The second syringe intended for intravenous/intraosseous delivery is labelled with black markings, as shown in FIGS. 8A-8B. While gray and black markings are shown, it will be understood that any color pairings, such as easily visually differentiable color pairings, are within the scope of the present disclosure. In some embodiments, the first color and the second color are particularly distinct from one another (such as yellow and red) so that a practitioner can easily distinguish the first and second syringes.

FIGS. 8A-8B illustrates an example of written indicia for administering a dose of a pharmaceutical composition for intravenous use, in accordance with an embodiment of the present disclosure. In some embodiments, the kit includes a second syringe configured for intravenous use, as shown in FIG. 9. In the illustrated embodiment, the written descriptions further include color coding corresponding to color markings on the first (FIGS. 7A-7B) and second syringe, providing further indicia of the appropriate dosage and delivery mechanism for a pharmaceutical composition. Such written description is suitable to ensure appropriate dosage and delivery mechanism to a health care provider who may be under time and other constraints/pressures in an emergent situation.

FIG. 8A is an example of instructions for an empty syringe. In some embodiments, the kit includes a second empty syringe configured to be filled with a pharmaceutical composition, such as epinephrine, and configured to be used intravenously. In some embodiments, the written indicia on the first syringe, configured for endotracheal use, differs from the written indicia on the second syringe, configured for IV/IO use. In some embodiments, the second syringe includes a female-to-female Luer coupler option to fill the second syringe more quickly. In some embodiments the female-to-female Luer coupler is couplable to the second syringe, but in other embodiments, the female-to-female Luer coupler is integrated into the second syringe. Illustrated is an example dosing chart, similar to that shown in Table 1, and an indicator requiring a user to use a specific concentration of epinephrine with the syringe. Although the example written indicia indicate that 0.1 mg/ml concentration of epinephrine is required for use with the first syringe, in some embodiments, any concentration of epinephrine suitable to administer a dose to a patient may be indicated.

FIG. 8B is an example of instructions for administering a pharmaceutical composition for intravenous use for a syringe that is prefilled. In some embodiments, the syringe is prefilled with a pharmaceutical composition. In some embodiments, the syringe is filed with 0.1 mg/ml concentration epinephrine. The prefilled syringe may have enough inside to administer a single dose to a neonate up to 5 kg, but in some embodiments, the prefilled syringe is prefilled with enough pharmaceutical composition for delivering the correct dose to a patient of any weight.

FIG. 9 schematically illustrates a kit 800 and an example of its use, in accordance with an embodiment of the present disclosure. In an embodiment, the syringe is a first syringe 805A, and the kit further comprises a second syringe 805B. As shown, the kit 800 includes two syringes: a first syringe 805A and second syringe 805B. In an embodiment, the first syringe 805A and the second syringe 805B are independently selected from the syringes and/or adaptors illustrated in and described further herein with respect to FIGS. 3A-3C, 4A-4C, and 5A-5C.

In an embodiment, the first syringe 805A and second syringe 805B are configured and suitable to deliver a pharmaceutical composition to a subject by different delivery mechanisms. In some embodiments, the first syringe 805A is configured to couple to an endotracheal tube, while the second syringe 805B is configured to couple to a syringe. In some embodiments, the kit includes a box 820 or other suitable packaging for holding the two syringes and any other components of the kit 800.

In some embodiments, the kit includes safety features such as labeling each syringe with the appropriate drug and concentration throughout the entire process of delivering the drug. In some embodiments, each syringe is color coded on the barrel and the end of the syringe, such as red for IV/IO and yellow for ETT, though it is appreciated that the syringes may be labeled with any color. While FIG. 9 is in black and white, the ETT syringe 805A is labeled “Yellow”, while the IV/IO syringe 805B is labeled “Red” to suggest a possible color combination of the two syringes 805A, 805B.

In an embodiment, a barrel size of the first syringe 805A is different than a barrel size of the second syringe 805B. Such a difference in barrel size between the first syringe 805A and the second syringe 805B can be useful in differentiating between uses for each syringe. As discussed further herein, in an embodiment, the first syringe 805A is suitable for use with an ETT. In an embodiment, the second syringe 805B is suitable for use with a needle or other structure configured for intravenous and/or intraosseous delivery of a pharmaceutical composition. Dosages for endotracheal delivery of a pharmaceutical composition are different than (and frequently larger than) dosages for intravenous/intraosseous delivery of the same pharmaceutical composition. Accordingly, it is useful in differentiating between the two syringes and their respective uses. A difference in barrel size between the first and second syringe is useful in this regard, as well as providing a capability of delivering a dosage of the pharmaceutical corresponding by the respective delivery mechanisms of the first and second syringe.

In an embodiment, components of the kit 800 include written indicia 825A, 825B, such as words or abbreviations, indicative of or denoting a use for components on which the written indicia are disposed. For example, in an embodiment, a syringe, such as the first syringe 805A, includes written indicia 825A including markings indicating that the syringe is for use with an endotracheal tube. In some embodiments, such written indicia 825A includes “ETT.” In an embodiment, the written indicia 825A, 825B further include markings indicating a dosage of a pharmaceutical composition when used with the endotracheal tube. Examples of such written indicia are illustrated in FIGS. 7A-7B and FIGS. 8A-8B. The markings indicating a dosage include markings indicating a range of weights of a subject to receive the pharmaceutical composition corresponding to the dosage of the pharmaceutical composition, such as those shown in FIGS. 7A-7B and 8A-8B. Such written indicia are useful in showing a user a position of a plunger of the syringe corresponding to a weight or estimated weight of a subject and a concentration of the pharmaceutical composition for use with the syringe. In an embodiment, the dosage is a neonatal dosage.

In a further embodiment, the second syringe 805B includes second written indicia 825B including second markings, such as indicating that the second syringe is for use with an intravenous or intraosseous delivery line. In an embodiment, the second written indicia 825B further includes markings indicating a dosage of the pharmaceutical composition when used with the intravenous or intraosseous delivery line, such as shown in FIGS. 8A-8B. In a further embodiment, the second markings indicating a dosage include markings indicating a range of weights of a subject to receive the pharmaceutical composition corresponding to the dosage of the pharmaceutical composition. In some embodiments, the color of the second written indicia 825B is distinct from the color of the first written indicia 825A, as illustrated in FIG. 9.

In an embodiment, the first written indicia 825A indicating a dosage are different than the second written indicia 825B indicating a dosage, as described in detail in FIGS. 7A-7B and 8A-8B. As discussed further herein, in certain embodiments, dosages for a pharmaceutical composition are different for endotracheal tube delivery and intravenous or intraosseous delivery. Marking the syringes accordingly is suitable to emphasize this difference in appropriate dosages.

In an embodiment, the kit 800 includes written instructions 810 for administering a dose of a pharmaceutical composition to a subject in need thereof. Such written instructions 810 can be in addition to and separate from written indicia 825A, 825B disposed on, for example, one or more syringes of the kit 800. As illustrated in FIG. 9, in some embodiments, the written instructions 800 may be on the inside of the lid of a box 820 containing the kit 800. While the illustrated instructions show dashed lines instead of numbers, the dashed lines are representative of the numbers shown in, for example, Table 1.

In an embodiment, the kit 800 includes a pharmaceutical composition for delivery to a subject in need thereof with the syringe. Such a pharmaceutical composition may be suitable for resuscitating a subject, such as a neonatal subject. In an embodiment, the pharmaceutical composition is the same pharmaceutical composition as indicated on the written indicia and/or the written instructions. In an embodiment, the pharmaceutical composition is selected from the group consisting of epinephrine, naloxone, atropine, vasopressin, lidocaine, and a combination thereof. In an embodiment, the pharmaceutical composition is epinephrine.

In an embodiment, the pharmaceutical composition is disposed within one or both of the first syringe and the second syringe 805A, 805B. In an embodiment, the pharmaceutical composition is disposed separately from the first and/or the second syringe 805A, 805B.

In operation, a practitioner can open the kit 800 in step #1. In some embodiments, the kit 800 contains two prefilled syringes 805A, 805B. The first syringe 805A may be an ETT syringe containing a single dose and the second syringe 805B may be an IV/IO syringe, containing a single dose. In step #2, the practitioner removes the desired syringe for the appropriate administration of the drug. In some embodiments, the appropriate administration includes IV/IO and ETT. In some embodiments, the kit 800 includes a recommended dosing chart (or instructions) 810 for quick reference on the top of the box 820 that contains the two syringes 805A, 805B. In step #3, the practitioner wastes the syringe to the appropriate weight that is marked, with written indicia 825A, 825B on either the ETT or IV/IO syringe. As discussed herein, in some embodiments, the ETT syringe 805A is a larger size to accommodate the larger required dose, and the IV/IO syringe 805B is smaller to accommodate the smaller required dose. In step #4,the practitioner administers the drug to the patient.

While illustrative embodiments have been illustrated and described, it will be appreciated that various changes can be made therein without departing from the spirit and scope of the invention.

The embodiments of the invention in which an exclusive property or privilege is claimed are defined as follows:

1. A kit comprising:
a syringe having a tip defining a male Luer taper fitting; and
an adaptor defining a first end portion shaped to couple with the male Luer taper fitting and a second end portion shaped to cooperatively couple with an opening of an endotracheal tube.

2. The kit of claim 1, wherein the first end portion of the adaptor comprises a female Luer taper fitting configured to cooperatively couple with the male Luer taper fitting, and wherein the second end portion comprises
a barrel portion shaped to couple to an outer surface of an endotracheal tube injection line; and
a tube portion positioned coaxially within the barrel portion and shaped to couple with an inner surface of the endotracheal tube injection line.

3. The kit of claim 1, wherein the second end portion is shaped to couple to an inner surface of the endotracheal tube injection line or cuff.

4. The kit of claim 1, wherein the adaptor is configured to transfer liquid between the syringe coupled to the first end portion and an endotracheal tube coupled to the second end portion.

5. The kit of claim 1, wherein the syringe is a first syringe, the kit further comprising a second syringe having a tip defining a male Luer taper fitting.

6. The kit of claim 5, wherein a barrel size of the first syringe is different than a barrel size of the second syringe.

7. The kit of claim 5, wherein the first syringe has a first color and the second syringe has a second color different than the first color.

8. The kit of claim 5, wherein the first syringe includes written indicia including markings indicating that the syringe is for use with an endotracheal tube.

9. The kit of claim 8, wherein the written indicia further includes markings indicating a dosage of a pharmaceutical composition when used with the endotracheal tube.

10. The kit of claim 9, wherein the markings indicating a dosage include markings indicating a range of weights of a subject to receive the pharmaceutical composition corresponding to the dosage of the pharmaceutical composition.

11. The kit of claim 9, wherein the dosage is a neonatal dosage.

12. The kit of claim 8, wherein the second syringe includes second written indicia including second markings indicating that the second syringe is for use with an intravenous or intraosseous delivery line.

13. The kit of claim 12, wherein the second written indicia further includes markings indicating a dosage of the pharmaceutical composition when used with the intravenous or intraosseous delivery line.

14. The kit of claim 13, wherein the second markings indicating a dosage include markings indicating a range of weights of a subject to receive the pharmaceutical composition corresponding to the dosage of the pharmaceutical composition.

15. The kit of claim 13, wherein the markings indicating a dosage are different than the second markings indicating a dosage.

16. The kit of claim 1, further comprising written instructions for administering a dose of a pharmaceutical composition to a subject in need thereof.

17. The kit of claim 16, wherein the method of administering the pharmaceutical composition can include administering the pharmaceutical composition through an endotracheal tube, through an intraosseous line, or through an intravenous line.

18. The kit of claim 1, further comprising a pharmaceutical composition for delivery to a subject in need thereof with the syringe.

19. The kit of claim 20, wherein the pharmaceutical composition is selected from the group consisting of epinephrine, naloxone, atropine, vasopressin, lidocaine, and a combination thereof.

20. The kit of claim 20, wherein pharmaceutical composition is disposed within the syringe.
